(19) United States
(12) Patent Application Publication
(10) Pub. No.: US 2008/0199396 A1
(43) Pub. Date: Aug. 21, 2008

(54) Radioisotope Labelled Biological Compositions, And Their Use In Accelerator Mass Spectrometry

(76) Inventors: Ronald Colin Garner, York (GB); Graham John Lappin, York (GB)

Correspondence Address: JACOBSON HOLMAN PLLC, 400 SEVENTH STREET N.W., SUITE 600, WASHINGTON, DC, 20004, US

(21) Appl. No.: 11/885,431

(22) PCT Filed: Feb. 28, 2006
(86) PCT No.: PCT/GB06/00713

§ 371 Date: Aug. 31, 2007

(30) Foreign Application Priority Data
Mar. 2, 2005 (GB) 0504243.7

(51) Int. Cl. A61K 51/10; C07K 14/00; A61K 51/08; G01N 33/53; C07K 16/00

(52) U.S. Cl. 424/1.49; 530/300; 530/387.1; 424/1.69; 435/7.2

(57) ABSTRACT

The invention provides a biological composition comprising a biological compound of formula I

B-A* (I)

alone or together with B
wherein B-A* is a biological compound or a derivative thereof formed as the product of reaction of the biological compound to introduce A*, A* is a radioisotopic moiety having MW in the range 10 to 500 comprising an AMS radioisotope * wherein the composition is characterised by a value for the percent incorporation of radioisotope which is a measure of maximum specific activity, wherein 100% incorporation is defined as the incorporation of one radioisotope per molecule, and wherein the percent incorporation is in the range from in excess of zero to 100%, wherein the quantity of organic radioisotope A* labelled molecules in a population of one thousand molecules is so low as to not alter the biological activity of the drug; a process for the preparation thereof; a method for AMS detection of one or more biological compositions as defined in any of claims 1 to 17 comprising B of same or different origin, comprising providing one or more biological compositions and optionally one or more control compositions comprising biological compound B, for dosing to at least one subject, obtaining metabolic samples from the subject(s) having been dosed with said biological(s) as hereinbefore defined and conducting AMS detection and obtaining AMS results for the or each biological; and the use of a biological composition or compound of formula I in AMS detection providing in vitro or in vivo metabolism characteristics thereof.

The present invention relates to a biological composition labelled with radioisotope, a biological compound of formula I labelled with radioisotope, a process for the preparation thereof, a method for AMS (accelerator mass spectrometry) detection of one or more samples derived from a subject dosed with one or more biological compositions; and the use of the biological composition in AMS detection providing in vitro or in vivo metabolism characteristics thereof.

There is a need to analyse biologicals which are being developed as drugs in order to (1) quantitate them and (2) detect their presence and metabolic fate in animal species especially humans. Recombinant antibodies, chemically synthesized peptides, oligonucleotides etc are all difficult to analyse using mass spectrometry, immunoassay, (for example ELISA immunoassays) or other methods such as fluorescence or UV absorption. This is particularly so when there is an endogenous equivalent to the biological drug. For example the number of antibody based drugs on the market and under development is rapidly increasing but the analysis of these after administration to human subjects is difficult owing to the high background of endogenous antibodies. Metabolic fate studies of these biologicals may prove crucial in understanding individual differences in response, why no biological action may be seen clinically despite high affinity binding to the target receptor in vitro. In addition it is known that glycosylation status can affect both clinical activity and metabolic stability. Furthermore small biological molecules such as Fabs are unstable in humans and so are stabilised through chemically bonding to a stabilising molecule such as polyethylene glycol. Again it is analytically challenging to quantitate blood levels of these stabilised molecules.

Moreover there is a need to determine bioequivalence of biological molecules derived from different sources.

We have now found that it is possible to employ Accelerator Mass Spectrometry (AMS) as a detection method to study binding to target, metabolic fate etc of a biological molecule. The attraction of the method is that it would be more sensitive and specific than current analytical methods such as ELISA and method development could be carried out in a relatively short time period.

AMS performs isotope quantification of isotopically labelled drugs in body fluids obtained from a human who have received radioactive doses. One of the most significant advantages of AMS is that it can detect and quantify with relatively short analytical times, levels of radioactivity that are so low that the dose needed to be administered to a human subject falls below the stipulated levels of radioactivity which require regulatory approval.

Labelling biological molecules with radioisotope tags is known, for example with ¹³¹I, ¹⁴C etc, by reacting the biological molecule with the isotope. Alternatively, ¹⁴C can be incorporated biosynthetically in radioactive culture. However this presents several problems, such as the labelled biological compound derived by such means typically has a radioactivity which exceeds that permitted by regulatory authorities for dosing to humans. Conversely, the radioactivity may be too low to allow detection by conventional methods eg Liquid Scintillation Counting. Where labels such as radio-iodine are used, these are gamma-emitters and therefore present special safety issues if administered to humans. Moreover, the half-life of ¹²⁵I is only 60.3 days and therefore useful life of the labelled compound is a limited.

A synthetic chemical approach to radioisotope labelling of biological compounds would not be expected to succeed. For example the formulation of proteins, reacting lysine with formaldehyde is a well known technique for fixing tissues, as it cross links the protein. Therefore generating radioisotope labelled derivatives of biological compounds is highly likely not only to damage the compound, but also affect its properties and activity.

We have now however found that it is possible to provide a radioisotope-labelled biological compound labelled with small molecular weight labelling reagents in a level of labelling sufficient for AMS detection of biological administered to a subject but not so great as to alter the biological activity or metabolic fate of the labelled biological.

Accordingly in the broadest aspect of the present invention there is provided a biological composition comprising a biological compound of formula I

B-A* (I)

alone or together with B

wherein B-A* is a biological compound or a derivative thereof formed as the product of reaction of the biological compound to introduce A*, A* is a radioisotopic moiety having MW in the range 10 to 500 comprising an AMS radioisotope * wherein the composition is characterised by a value for the percent incorporation of radioisotope which is a measure of maximum specific activity, wherein 100% incorporation is defined as the incorporation of one radioisotope per molecule, and wherein the percent incorporation is in the range from in excess of zero to 100%.

In a particular advantage the quantity of radioisotope A* labelled molecules in a population of molecules thereof is so low as to not alter the biological activity of the compound or composition, having regard to that of biological compound B.

In a particular advantage we have found that a biological composition of the invention may be prepared by known and novel means without the damage to the biological incurred with prior art methods employing a greater percent incorporation of label. However the radioisotope is present in an amount which is within the limits of AMS detection.

Reference hereinafter to a lightly labelled biological is to the biological composition or compound comprising radioisotope present in amount detectable by AMS, preferably corresponding to a value for percent incorporation in a range as hereinbefore defined. Percent incorporation is proportional to maximum specific activity, wherein 100% incorporation is defined as the incorporation of one radioisotope per molecule, i.e. every mole of substance contains a mole of the radioisotope.

An AMS radioisotope as hereinreferred may be any isotope which is relevant or susceptible to AMS analysis. AMS radioisotopes preferably have very low natural backgrounds, for example in the range from 1×10⁻⁵% or less for example to 1×10⁻¹⁵%, and artificially available isotopes have zero natural abundance. The sensitivity of AMS relies on the fact that AMS radioisotopes have a very low natural background such as approximately 1.4×10⁻¹⁰% for ¹⁴C. The background for ¹³C is 1.1%, which by comparison is huge. This means that the incorporation rate for ¹³C would have to be much higher than for ¹⁴C. Preferably AMS radioisotopes have long half lives in excess of weeks up to 1,000's of years for ease of handling. Preferably AMS radioisotopes are non-toxic in AMS levels, whereby they are suitable for human metabolism, and preferably are of biomedical interest.

Preferably percent incorporation is fractional in the range of in excess of zero to 100%, for example in the range 1×10⁻¹² to 5%, more preferably 0.1 to 2%. The invention therefore takes advantage of the analytical power of AMS and the fact that AMS can uniquely be applied to trace radioisotope labelled biologicals.

Percent incorporation can be derived directly or calculated from the specific activity of the biological B. The maximum specific activity is given in dpm/mmole and is the basic unit of radioactivity—disintegrations per minute—being the number of nuclear disintegrations occurring, on average, every minute. However percent incorporation is a normalised function which can be compared for all radioisotopes, and is more instructive than the term of specific activity which is dependent on radioisotope.

Preferably therefore % incorporation for a biological composition of the invention is determined by equation Equ 1:

$$\% incorporation ( m ) = \frac{100 \times specific activity}{maximum specific activity} \qquad Equ 1$$

For any radioisotope (based on one radioisotope per molecule, corresponding to 100% incorporation) the maximum specific activity is given by the equation Equ 2:

$$\frac{ln 2}{{t}_{1 / 2}} \times N \qquad Equ 2$$

where ln2 is the natural log of 2 (=0.6932)

t_{1/2} is the half-life of the radioisotope in minutes

N is the number of molecules in 1 mmole of labelled biological composition (moles biological×6.0225×10²⁰).

(Lappin, G and Garner, R C (2003) Ultra sensitive detection of radiolabelled drugs and their metabolites using Accelerator Mass Spectrometry. Chapter 11 in: Wilson, I D (ed) Bioanalytical Separations Handbook of Separations Vol 4. Elsevier Science BV Amsterdam)

Equation 2 takes no account of diminishing radioactivity (dpm value) due to the half-life of the radioisotope over time (ie it calculates the maximum specific activity at time zero).

The following example calculates the maximum specific activity based on one radioisotope per molecule) for ¹⁴C. The half-life of ¹⁴C is 5730 years (5730×365.3×24×60 minutes) and so any diminishment of radioactivity over short periods of time is negligible. In the following illustration units are as follows:

Bq=Becquerel=60 dpm

kBq: 60×10³ dpm

GBq: 60×10⁹ dpm

kBq/mmole: maximum theoretical specific activity (based on one radioisotope per molecule).

mmole=10⁻³ mole

amole=10⁻¹⁸ mole

From equation Equ 2:

$$\begin{matrix}\frac{0.6932}{( 5730 \times 365.3 \times 24 \times 60 )} \times 6.0225 \times {10}^{20} = 1.385 \times {10}^{11} dpm \\ = 2.3083 \times {10}^{6} kBq / mmole\end{matrix}$$

Thus, the maximum theoretical specific activity for ¹⁴C is 2.3083 GBq/mmole, (based on one radioisotope per molecule). This equals 100% incorporation.

Percentage incorporation for lightly labelled biologicals according to the present invention is therefore suitably determined by the above equation Equ 1:

Taking a biological B of 160,000 molecular weight, for 100% incorporation the specific activity would be 2.3 GBq/160,000 mg or 6.8 dpm/ng (see above calculation). As a general rule AMS can detect 0.06 dpm/mL plasma, thus putting the assay in the fg range. Thus if the rate of incorporation of ¹⁴C was only 1%, this would place the assay in the picogram range (low pg range of approximately 100 pg). This is more than enough sensitivity for an AMS measurement as the limit of detection for a typical ELISA assay would be an order of magnitude higher. (ng=10⁻⁹, pg=10^{-12,} fg=10⁻¹⁵ g)

B is suitably of molecular weight MW in excess of 1000, for example from 1000 up to 5 million or more. Many biologicals lie within the range 1,000 to 200,000 Daltons, for example in the range 50,000 to 200,000 Daltons.

B may be selected from any class of biological which it is desired to investigate in connection with the human or animal body or plants or soil matter and may be a natural or synthetic biological compound, for example selected from polysaccharides, biopolymers, amino acids, proteins, peptides, oligonucleotides, nucleic acid, RNA, DNA, fatty acids, carbohydrates, insulin analogues, growth hormone analogues, plant or gene therapy products and the like, for example antibodies such as recombinant antibodies, GH-recombinant antibodies and the like, erythropoietin and the like. Preferably B is a medicament, veterinary product or agrochemical, or a candidate therefor, for use in treatment of the human or animal body or plant.

The radioisotopic moiety A* is preferably selected from an organic moiety such as ¹⁴C or ³H C₁₋₄ alkyl, alcohol, ether, and the like, for example methyl (formyl), hydroxymethyl, hydroxyethyl and the like or an unconjugated isotope such as an unconjugated ³⁶Cl isotope.

In the case that the composition comprises biological compound of formula I as hereinbefore defined, the radioisotopic moiety A* is characterised by a percent incorporation corresponding to that of the compound of formula I as hereinbefore defined. Alternatively in the case that the composition comprises biological compound of formula I together with biological compound B as hereinbefore defined, the radioisotopic moiety A* is characterised by a percent incorporation greater than that of the compound of formula I as hereinbefore defined.

A radioisotope * is suitably selected from any radioisotope which is amenable to detection by AMS detection techniques. Radioisotopes vary in half life and thereby in radioactivity and enable detection in smaller or greater amounts whereby certain radioisotopes are particularly suited for certain envisaged applications either by virtue of the chemical nature of the isotope or its radiation characteristics. All atoms have isotopic forms, some of which are suited to AMS analysis. For example a biological labelled with ¹²⁹I is useful for AMS detection whereas a biological labelled with ¹³¹I although highly active is probably of limited use in humans due to safety issues. Similarly a biological labelled with ¹⁴C is useful for AMS detection whereas a biological labelled with ¹³C is of widespread use in many other non-radioactive techniques, such as NMR detection, for example as disclosed in WO 97/01098, but of no use in AMS analysis. Particularly unsuitable isotopes fail to form negative ions, notably nitrogen.

A biological composition of the invention therefore suitably comprises AMS radioisotope selected from AMS radioisotopes of hydrogen, beryllium, carbon, aluminium, phosphorus, chlorine, calcium, manganese, iron, selenium, iodine, barium and lanthanides and actinides such as uranium or plutonium. Preferably a biological composition of the invention is radioisotope labelled with an isotope selected from any one or more of ³H, isotopes of Ba, ⁷Be, ¹⁰Be, ¹⁴C, ¹⁷O, ¹⁸O, ²⁶Mg, ²⁶Al, ³²Si, ³⁵S, ³⁶Cl, ⁴¹Ca, ⁵⁵Fe, ⁶⁰Fe, ⁵³Mn, ⁷⁹Se ⁵⁹Ni, and ¹²⁹I, most preferably selected from any one or more of ³H, ¹⁴C and ³⁶Cl.

In a preferred embodiment the invention comprises a biological composition as hereinbefore defined characterised in that an AMS radioisotope is a ¹⁴C radioisotope; alternatively or additionally a ³⁶Cl or³H radioisotope. A biological composition of the invention may comprise more than one radioisotope which may be the same or different, and are preferably different.

In a further aspect of the invention there is provided a biological compound of formula I

B-A* (I)

wherein B-A* is the product of reaction of a biological compound B to introduce A*, A* is a radioisotopic moiety having MW in the range 10 to 500 comprising an AMS radioisotope *, wherein the compound is characterised by a value for the percent incorporation of radioisotope which is a measure of maximum specific activity, wherein 100% incorporation is defined as the incorporation of one radioisotope per molecule, and wherein the percent incorporation is in the range from in excess of zero to 100%.

Preferably the compound of the invention is characterised by the same features and advantages as the composition of the invention as hereinbefore defined. For example, preferably percent incorporation is fractional in the range of in excess of zero to 100%, for example in the range 1×10⁻¹² to 5%, more preferably 0.1 to 2%.

In a further aspect of the invention there is provided a process for the preparation of a biological composition as hereinbefore defined comprising chemically labelling an amount thereof with a moiety A* as hereinbefore defined such that the percent incorporation of AMS radioisotope * is in the range from in excess of zero to 100% preferably fractional in the range of in excess of zero to 100%, as hereinbefore defined. Preferably such labelling is carried out ex vivo. Similarly, this process may be used to prepare a compound of the invention, as hereinbefore defined.

Preferably a chemical labelling process for the preparation of a biological composition as hereinbefore defined comprises reacting a biological compound B as hereinbefore defined

with a reactive agent of formula II or III

A′* (II)

XₙA* (III)

wherein B, A and * are as hereinbefore defined, A′ is a reactive precursor to A, n is 0 or 1 and X is a leaving group

wherein agent of formula II or III is characterised by a percent incorporation of radioisotope corresponding to the desired percent incorporation of the composition as hereinbefore defined, or in excess thereof,

and in the case that agent of formula II or III is characterised by a percent incorporation of radioisotope corresponding to the desired, obtaining product composition as hereinbefore defined,

or alternatively in the case that agent of formula II or III is characterised by a percent incorporation of radioisotope in excess of that desired, obtaining a compound of formula I having percent incorporation of radioisotope in excess of that as hereinbefore defined, and additionally combining the obtained compound of formula I with an amount of B and obtaining product composition as hereinbefore defined.

One particularly preferred chemical labelling process involves the reaction of a biological compound B, as hereinbefore defined, in particular a protein, preferably of therapeutic benefit to humans, to attach a radioisotope moiety A* thereto.

In all aspects of this invention, A* is a radioisotopic moiety having MW in the range 10 to 500. Preferably this range is from 10 to 200, more preferably from 30 to 150. Preferably A* contains a single ¹⁴C atom.

We particularly refer that the labelling process is a so-called conjugation method whereby the AMS radioisotope * is attached to a chemical species which chemical species is then reacted with the biological compound B.

There is a wide range at conjugating reagents commercially available and the skilled person will be aware how to manufacture corresponding reagents which incorporate an AMS radioisotope. Such AMS radioisotope-containing conjugates may and preferably are to be used, in the practise of the chemical labelling process of this invention.

An example of a useful class of conjugating reagents is the maleimides, which react with thiols at pH<7 and with amines at pH around 9-10 schematically represented in the Scheme below:

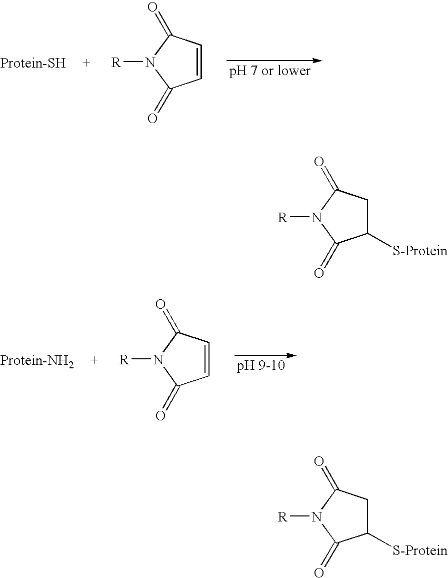

A particularly convenient maleimide is the commercially available compound ¹⁴C-N-ethyl maleimide.

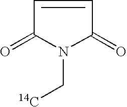

Another useful class of conjugating reagents is the active esters, which can be used to react with free amine moieties. The term “active ester” is a term which is well understood by the skilled person and refers to an ester R′C(O)OR in which R is a group other than an alkyl group which serves to increase the electrophilicity of the ester carbonyl functionality. The skilled person also knows that the electrophilicity of an ester may be tailored by incorporating particular OR groups; for example the Bolton-Hunter reagent, and modifications thereof discussed herein is susceptible to nucleophilic attack by free amine groups. A particularly preferred class of active ester is related to the Bolton-Hunter reagent (Bolton, A. E. and Hunter, W. M., The labelling of proteins to high specific radioactivites by conjugation to a ¹²⁵I-containing acylating agent, Biochem J 133 (3), 529-39, 1973). According to the invention, a reagent structurally related to the Bolton-Hunter reagent may be prepared in which the ¹²⁵I atoms in the reagent are absent. Instead, as the means for detection, an AMS active isotope, such as a ¹⁴C atom contained within the phenyl ring of the reagent is present.

Preferably a compound of formula II or III is a functionalised or unfunctionalised C₁₋₄ hydrocarbon wherein A′ or A is ¹⁴C or ³H C₁₋₄ hydrocarbyl or an unconjugated radioisotope and X is selected from —C═O, —Br, —I, —Cl, —OH and the like or is an unconjugated atom. Preferably a compound of formula II or III is ¹⁴C or ³H alkene, formaldehyde, acetaldehyde or any other methylating agent as known in the art or is ³⁶Cl chlorine gas (Cl₂).

In a particular advantage of the invention the labelling by a moiety A* as hereinbefore defined of a material is such as to not alter the biological activity thereof. Preferably chemical labelling of GMP material and the like would not affect its GMP status providing labelling is carried out on the GMP conditions. In a further advantage we have found that lightly labelling a biological as hereinbefore defined is conducted at such a low level that there is no destruction of the biological itself, for example cross linking or the like by formaldehyde.

Preferably therefore the process comprises labelling an agent II or III or labelling a compound B to give a compound of formula I, determining the specific activity thereof, determining the desired specific activity to give a desired percent incorporation, and combining with a sufficient amount of corresponding unlabeled agent or compound of formula I or biological B and isolating as a homogeneous product or as a composition having desired percent incorporation as hereinbefore defined.

In a further aspect of the invention there is provided a method for AMS detection of one or more biological compositions of the invention as hereinbefore defined comprising B of same or different origin, comprising providing one or more biological compositions and optionally one or more control compositions comprising biological compound B, for dosing to at least one subject, obtaining metabolic samples from the subject(s) having been dosed with said biological(s) as hereinbefore defined and conducting AMS detection and obtaining AMS results for the or each biological.

Preferably the method comprises in a first stage providing a sample of biological B from each source and conducting the process of the invention as hereinbefore defined for the preparation of a biological composition as hereinbefore defined. It will be understood that the conducting of the process of the invention in this way is ex vivo conducting. A subject may be any human, animal or plant or may be an assay medium or cell culture.

In a first embodiment of the invention the method for AMS detection is a method for detecting one biological composition for the purpose of determining binding to target, metabolic fate and the like. The skilled operator is able to interpret the AMS results and derive information on the efficacy of the biological composition, its toxicology, pharmacokinetics, metabolic fate and the like.

In a second embodiment of the invention the method is a method for determining the bioequivalence of two or more biologicals B derived from same or different origin and is a method for product control and establishing reproducibility of the source of biological B, or is a method for determining that one or more biologicals B derived from one or more different sources are bioequivalent to a biological B from a first source, in each case by conducting AMS on their corresponding compositions as hereinbefore defined. Preferably therefore the method comprises the additional step of comparing AMS results for each biological B with its corresponding composition.

Bioequivalence, as is known to those skilled in the art, is a measure of the bioavailabilty of one drug relative to another. Moreover it has specific regulatory guideline associated with it (EMEA Note for guidance on the investigation of bioavailability and bioequivalence London 26 Jul. 2001 CPMP/EWP/QWP/1401/98) and all references herein to bioequivalence are to be understood to be defined in accordance with this guideline.

Preferably the one or more biologicals B derived from different sources are generic biologicals which are intended to be used for the same purpose as a known proprietary biological, typically as a medicament, animal or plant health product.

The method of the invention is useful both in providing for in vitro or in vivo activity, reactivity, inhibition or functionality screening and selection of biologicals B, binding or metabolic data for biologicals B, in particular for providing ADME and PK data.

AMS dosing is suitably by administering an amount of lightly labelled biological alone or with a suitable carrier to a human or animal subject. Administration is typically by oral, dermal, buccal, vaginal, anal, subcutaneous, nasal, intravenous, ocular route or by inhalation. A dose suitably comprises sufficient biological to give a low dose of the order of nanocuries of radioactive label, for example is of the order of ng to mg. Preferably a dose is less than 1 microSievert, thereby being exempt from regulatory approval. A dose may therefore comprise from 1 nanogram to 1 milligram, preferably 1 microgram to 500 micrograms of lightly radioisotope labelled biological of the invention.

After a period of days, weeks or months, samples are taken of tissue or cells, blood samples, urine or faeces, expired air or the like. Samples are suitably taken at intervals in order to detect biological metabolism rate and indicate rapid and slowly metabolised biologicals. The method is described in WO 01/59476, the contents of which are incorporated herein by reference. Analysis of AMS results indicates number of isotope counts, eg of ¹⁴C, ratio of modern (ie naturally occurring) isotopes and percent modem isotope as a combination of the number of counts and the ratio of modem isotope. pMC (percent modem carbon) is an AMS term of radioactivity and provides a measure of the carbon content of a sample. pMC=Times modern×100. One times modern=¹⁴C/¹²C ratio in the atmosphere in 1952. The ratio ¹²C/¹³C remains relatively constant.

A sample is preferably prepared for AMS from any sample which is derived from an assay, such as a cell or cell membrane sample, or from human, animal or plant derived dosing samples, such as tissues or cells, bodily fluids such as blood or urine, faeces, plant tissues, soil or soil organisms such as worms and the like.

In the method of the invention a sample is prepared for AMS analysis in a range of micrograms or less of tissues or cells to a few microlitres of blood or urine. Samples may also comprise plant tissues, soil or soil organisms such as worms, as known in the art.

The sample is prepared in a form that can yield negative ions within the instrument's ion source, as known in the art. Sample preparation may be by traditional methods which prepare thermally and electrically conductive solids, are non-fractionating, efficient and protected from contamination by isobars or unexpected concentrations of the rare isotope in or on laboratory equipment. Uniformity and comparability between samples and standards are ensured by reducing all samples to a homogeneous state from which the final target material is prepared. Reduced sample is then compressed into tablet form in a cylindrical aluminium cathode before elemental isotope ratio analysis in the AMS.

For example samples obtained from dosing isotopic carbon labelled biological compositions of the invention may be converted to graphite, samples obtained from microdosing isotopic halide labelled biological compositions of the invention may be converted to silver halide salts, samples obtained from microdosing isotopic aluminium labelled biological compositions of the invention may be converted to aluminium oxide and samples obtained from microdosing isotopic calcium labelled biological compositions of the invention may be converted to a calcium dihalide or dianhydride. Conversion is for example performed for carbon samples (containing ¹⁴C) by oxidising to CO₂ before reducing to graphite, commonly by the reduction of the CO₂ by hydrogen or zinc over an iron or cobalt catalyst or binder (Vogel J S (1992) Rapid production of graphite without contamination for biomedical AMS, Radiocarbon, 34, 344-350). Oxidation is in a sealed tube which is heated in a furnace at temperatures of up to 900 C with an oxidant such as copper oxide for approx 8 hours. The resulting CO₂ is reduced to graphite in a second step after cryogenic transfer using a reducing agent such as zinc and titanium hydride and cobalt as a catalyst at temperatures up to about 500 C for approx 18 hours with cooling. Cobalt/graphite is then compressed into tablet form in a cylindrical aluminium cathode before elemental isotope ratio analysis in the AMS.

Alternatively sample preparation may be for example by the improved technique of WO 01/59476, the contents of which are incorporated herein by reference. Preferably according to the method of WO 01/59476 sample is homogeneously mixed with a binder which is preferably electrically conductive and may be any substance which allows the mixture of sample and binder to be compressed into tablet form. More preferably the binder is one or a mixture of any of graphite, cobalt or aluminium powder, for example where the isotope to be detected is ¹⁴C, or is one or a mixture of any or aluminium oxide and iron or iron oxide, for example where the isotope to be detected is plutonium.

In a particular advantage of this embodiment of the invention it is usually not possible to prepare a radioisotope labelled biological comprising a proprietary third party biological including one or more radioisotopes by culturing a proprietary cell line in radioactive culture, since the proprietary cell line is usually not available. For example the exclusivity of a proprietary biological medicament may be maintained by maintaining the exclusivity of the cell line from which it is derived. The method of the invention enables the preparation of a radioisotopically labelled analogue thereof by chemical synthetic means but so as to not alter the biological activity thereof, enabling direct comparison with a generic equivalent, and the determination of bioequivalence.

In this case the skilled AMS operator is able to interpret the AMS results and determine whether these are identical, and if there are any differences whether these are the consequence of bio-nonequivalence or of sample or host variation which are irrelevant to bio equivalence.

Alternatively it is not possible to synthesise radioisotope labelled endogenous human or animal biologicals if the synthesis of the biological itself is not available. The method of the invention provides for AMS determination of an endogenous human or animal biological B or bioequivalence of endogenous and genetically engineered biologicals B.

Alternatively the method may be for determining the non-bioequivalence of a competitive generic biological B with a proprietary biological B to ensure that the generic is not wrongly substituted for the proprietary, as a generic biological or as a pirate biological.

In a further aspect of the invention there is provided the use of a biological composition of the invention in AMS detection providing in vitro or in vivo metabolism characteristics thereof. Preferably the method is in dosing a subject and deriving samples for AMS detection using a method as hereinbefore defined. Dosing and AMS detection are known in the art, for example in WO 01/59476, the contents of which are incorporated herein by reference. A subject may be any human, animal or plant, as hereinbefore defined.

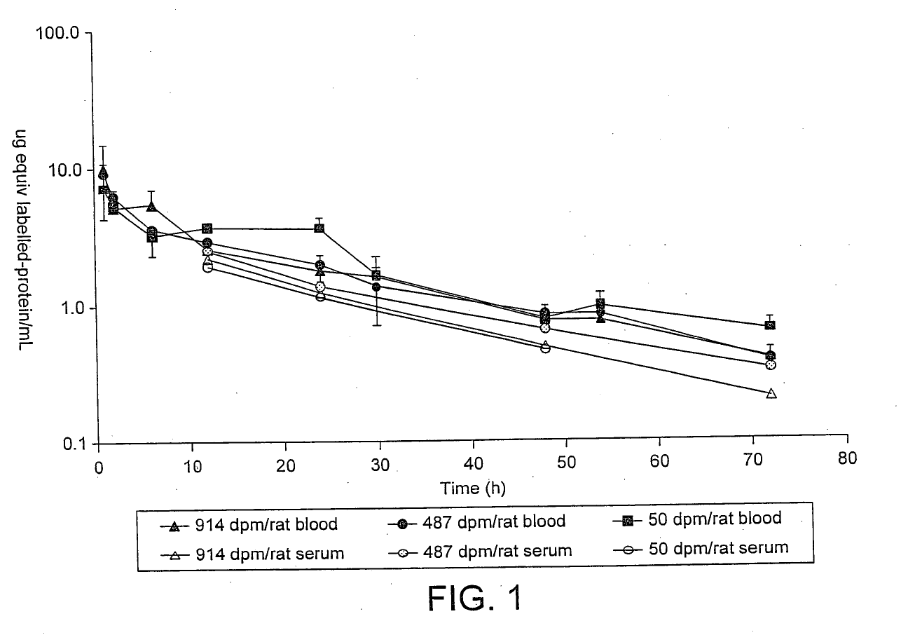

FIG. 1 is a graph showing that it is possible to measure protein concentration in rats by AMS previously administered with as little as 50 dpm of ¹⁴C-NEM-labelled human serum albumin (HSA).

The invention is now illustrated in non-limiting manner with reference to the following examples.

### EXAMPLE 1

### Demonstration That Biological Activity of a Biological B is Unaffected by the Process of the Invention

Antibody of approx 150,000 MW is ¹⁴C labelled to provide biological compositions of the invention in two different levels of percent incorporation. The compositions are assayed for biological activity and the results show that the biological activity is unaffected. Unlabelled antibody is used as a control, and shows that biological activity is unaffected.

### EXAMPLE 2

### Demonstration That Biological Activity of a Biological B is Unaffected by the Process of the Invention

The samples of Example 1 are dosed to a subject and AMS samples obtained. AMS detection is performed on the samples and results obtained. The results show the metabolic stability is unaffected.

### EXAMPLE 3

### Demonstration that Biological Activity of a Biological B is Unaffected by the Process of the Invention

In this study, an AMS isotope-containing moiety was covalently bound to a protein. The labelled protein was administered intravenously to rats, then blood and serum was sampled over time. The blood and serum samples were analysed by AMS to determine the protein concentration at each time point.

Methods

Protein Labelling

Human serum albumin (HuSA) was purchased in recombinant form from SeraCare, USA (purity >98%). HuSA is a well-characterised protein with a molecular weight of 66,500, containing 35 cysteinyl residues, only one of which (residue 34) is in the free reduced form [1]. Maleimides are known to react with free sulphydryls [2], therefore ¹⁴C—N-ethyl-maleimide (Amercian Radiochemicals) was reacted with HuSA for 6 hours at 37° C. at a ratio of 1 mole ¹⁴C—NEM to 1 mole HuSA. (In this example, ¹⁴C was the “AMS isotope”). The reaction mixture was in Phosphate Buffered Saline (pH 7). Following the reaction, the ¹⁴C-NEM-HuSA product was purified using a Ultra centrifugal filter device (Amicon Ultra-4, 10,000 molecular weight, supplied by Millipore, UK). The filtrate (protein) was dissolved in physiological saline. The ¹⁴C-labelled protein was diluted with non-labelled protein to achieve three solutions with specific activities of 3785, 1892 and 189 disintegrations per minute (dpm) per mg respectively. The molar ratio of the reaction was ¹⁴C-NEM to 20 HuSA.

Analysis of ¹⁴C-NEM-HuSA

A sample of the ¹⁴C-NEM-HuSA product was subjected to tryptic digestion and analysis with MALDI-TOF using a standard methodology applicable to protein analysis eg [3]. Total protein was determined as described in [4].

Animals and Dosing

Each ¹⁴C-labelled drug was administered to 3 groups of 4 Sprague Dawley Crl: CD (SD) rats (mean bodyweight 242 g) each group receiving one of the proteins at different specific activities. All doses were administered intravenously at a dose rate of 1 mg/kg bodyweight (approximately 0.5 mL administered per animal). The amount of radioactivity administered for each dose group was 914, 486 and 50 dpm per animal respectively.

Prior to dosing, animals were acclimatised for a minimum period of five days. Prior and following dosing animals were group housed and kept in rooms thermostatically maintained at a temperature of 19 to 25° C., with a relative humidity of between 40 to 70%, and exposed to fluorescent light on a cycle of 12 hours light (0600 to 1800)/12 hours dark.

Blood and Serum Sampling

Samples of blood (nominally 75 μL) were withdrawn from each animal, via a lateral tail vein, at each of the following times after dose administration: 1, 2, 6, 12, 24, 30, 48, 54 and 72 hours. Additional samples of blood (nominally 500 μL) were withdrawn at 12, 24, 48 and 72 hours. Blood was collected into non-heparinised tubes. The 500 μL samples were allowed to clot and centrifuged to prepare serum. Blood and serum were frozen rapidly after collection and stored at −60 to −80° C. until analysis.

Sample Analysis

All blood and serum samples were analysed to determine the ¹²C:¹⁴C ratio (reported as percent modern carbon) by AMS as previously described [5]. The results were converted to dpm/mL blood or serum [6] and then to ng equivalents of protein from the specific activity of the protein administered.

Results and Discussion

The tryptic digest and MALDI-TOFF indicated that ¹⁴C-NEM was covalently bound to the free sulphydryl group at residue 34. The MALDI-TOFF determines the molecular weight of protein fragments produced by the tryptic digest. NEM adds approximately 125 to the mass and a fragment with a molecular ion of 2558.36 was observed, corresponding to the attachment of NEM to the cysteine residue in the amino acid sequence:

| | |
|---|---|
| ALVLIAFAQYLQQCPFEDHVK. | (SEQ I.D. No 1) |

The concentration of ¹⁴C-NEM-HuSA was determined in all samples of whole blood and serum by AMS. It was possible to measure the protein concentration in samples derived from rats administered just 50 dpm of labelled protein (FIG. 1). To put this into context, in a conventional study of this type, the radioactive dose would be approximately 3×10⁷ dpm per animal. The background level of radioactivity in most conventional laboratories is around 50 dpm. The study demonstrates that using labelling technique attaching an “AMS isotope” to a relatively large molecular weight compound ex vivo, then allowed the compound to be followed in a biological system in vivo. In this particular example, the biological system was the rat but the concept has been equally proven for any animal species, including human. The levels of ¹⁴C used were far too small to be detected by conventional radioactive measurement methods and only AMS has the required sensitivity.

FIG. 1. HSA concentration (μg/mL) in rat blood and serum over time following administration of 1 mg/kg ¹⁴C-labelled HuSA. Protein concentration was measured using AMS analysis of a ¹⁴C-label on the HuSA. Error bars are standard deviation and n=4.

It will be evident to those skilled in the art that HSA represented a tough test for the methodology as it contains only one free SH group on which to add a label. In addition, it has been demonstrated that the labelled protein can be measured in both serum and whole blood. Conventional methods of protein determination such as ELISA can only be used with serum.

### REFERENCES

1. Ikegaya, K., M. Hirose, T. Ohmura, and K. Nokihara, Complete determination of disulfide forms of purified recombinant human serum albumin, secreted by the yeast Pichia pastoris. Anal Chem, 1997. 69(11): p. 1986-91.

2. Wilbur, D. S., Radiohalogenation of proteins: an overview of radionuclides, labeling methods, and reagents for conjugate labeling. Bioconjug Chem, 1992. 3(6): p. 433-70.

3. Tie, J. K., V. P. Mutucumarana, D. L. Straight, K. L. Carrick, R. M. Pope, and D. W. Stafford, Determination of disulfide bond assignment of human vitamin K-dependent gamma-glutamyl carboxylase by matrix-assisted laser desorption/ionization time-of-flight mass spectrometry. J Biol Chem, 2003. 278(46): p. 45468-75.

4. Mohammedi, H., S. Mamouzi, C. Allal, M. Ghaffor, H. Rabhi, and M. C. Abbadi, Rapid and sensitive micromethod for protein determination by the Coomassie-blue technique. Arch Inst Pasteur Alger, 1989. 57: p. 151-62.

5. Sarapa, N., P. H. Hsyu, G. Lappin, and R. C. Garner, The application of accelerator mass spectrometry to absolute bioavailability studies in humans: simultaneous administration of an intravenous microdose of 14C-nelfinavir mesylate solution and oral nelfinavir to healthy volunteers. J Clin Pharmacol, 2005. 45(10): p. 1198-205.

6. Lappin, G. and R. C. Garner, Ultra-sensitive detection of radiolabelled drugs and their metabolites using accelerator mass spectrometry, in Handbook of Bioanalytical Separations, I. Wilson, Editor. 2003, Elsevier Science: Amsterdam. p. 331-349.

Further aspects of the invention will be apparent from the foregoing.

1-38. (canceled)

39. A biological composition comprising a biological compound of formula I:
B-A* (I)
alone or in combination with B;
wherein B-A* is a biological compound, or a derivative thereof, formed as the product of reaction of the biological compound B to introduce A*, and A* is a radioisotopic moiety having MW in the range 10 to 500 having an AMS radioisotope *, wherein said composition also having a percent incorporation of radioisotope in the range of 1×10⁻¹² to 5%.

40. The biological composition of claim 39, wherein the percent incorporation is fractional in the range from 0.1 to 2%.

41. The biological composition of claim 39, wherein B-A* is formed as the product of a ex vivo chemical reaction between the biological compound B and a precursor to A*.

42. The biological composition of claim 39, wherein A* is a radioisotope moiety having MW in the range of 10 to 200.

43. The biological composition of claim 39, wherein the quantity of radioisotope A* labeled molecules in a population of molecules thereof is so low as to not alter the biological activity of the compound or composition, having regard to that of biological compound B.

44. The biological composition of claim 42, wherein the half life of the AMS isotope is in excess of weeks.

45. The biological composition of claim 39, wherein B has a molecular weight MW in excess of 1000.

46. The biological composition of claim 39, wherein B has a molecular weight MW in a range of 1000 to 5 million.

47. The biological composition of claim 45, wherein B is selected from the group consisting of polysaccharides, biopolymers, amino acids, proteins, peptides, oligonucleotides, nucleic acid, RNA, DNA, fatty acids, carbohydrates, insulin analogues, antibodies, hormones or hormone analogues.

48. The biological composition of claim 47, wherein B is a medicament, veterinary product or agrochemical, or a candidate therefor, for use in treatment of the human or animal body or plant.

49. The biological composition of claim 39, wherein A* is selected from an organic moiety such as ¹⁴C or ³H C₁₋₄ alkyl, alcohol, ether, and the like, for example methyl (formyl), hydroxymethyl, hydroxyethyl and the like or an unconjugated isotope such as an unconjugated ³⁶Cl isotope.

50. The biological composition of claim 49, wherein A* is derived from an active ester or from a maleimide.

51. The biological composition of claim 39, having a radioisotope * of hydrogen, beryllium, carbon, aluminium, phosphorus, chlorine, calcium, manganese, iron, selenium, iodine, barium and lanthanides and actinides such as uranium or plutonium.

52. The biological composition of claim 51, wherein said radioisotope * is selected from at least one or more of ³H, isotopes of Ba, ⁷Be, ¹⁰Be, ¹⁴C, ¹⁷O, ¹⁸O, ²⁶Mg, ²⁶Al, ³²Si, ³⁵S, ³⁶Cl, ⁴¹Ca, ⁵Fe, ⁶⁰Fe, ⁵³Mn, ⁷⁹Se ⁵⁹Ni, and ¹²⁹I.

53. The biological composition of claim 51, wherein said radioisotope * is selected from at least one or more of ³H, ¹⁴C and ³⁶Cl.

54. The biological composition of claim 39, wherein A* is derived from ¹⁴C—N-ethyl maleimide.

55. A biological compound of formula I
B-A*; (I)
wherein B-A* is the product of reaction of a biological compound B to introduce A*, and A* is a radioisotopic moiety having a molecular weight MW in the range 10 to 500 comprising an AMS radioisotope *, and wherein the percent incorporation is in the range of 1×10⁻¹² to 5%.

56. The biological composition of claim 55, wherein the percent incorporation is fractional in the range of 0.1 to 2%.

57. The biological composition of claim 55, wherein B-A* is as defined as the product of a ex vivo chemical reaction between the biological compound B and a precursor to A*.

58. The biological composition of claim 55, wherein A* is a radioisotope moiety having MW in the range of 10 to 200.

59. A process for the preparation of a biological composition of claim 39, comprising the steps of: chemically labeling an amount of said composition with a moiety A*, wherein A* is a radioisotopic moiety having a molecular weight MW in the range 10 to 500 comprising an AMS radioisotope *, and wherein the percent incorporation is in the range of 1×10⁻¹² to 5%.

60. The process of claim 59, wherein said percent incorporation is fractional in the range of 0.1 to 2%.

61. The process of claim 59, wherein said labeling is ex vivo labeling.

62. The process of claim 59, comprising the steps of: reacting a biological compound B, wherein B has a molecular weight MW in excess of 1000 and B is selected from the group consisting of polysaccharides, biopolymers, amino acids, proteins, peptides, oligonucleotides, nucleic acid, RNA, DNA, fatty acids, carbohydrates, insulin analogues, antibodies, hormones or hormone analogues;
with a reactive agent of formula II or III:
A′* (II)
XₙA* (III)
wherein A* is a radioisotopic moiety having a molecular weight MW in the range 10 to 500 comprising an AMS radioisotope *, and wherein the percent incorporation is in the range of 1×10⁻¹² to 5%,
and A′ is a reactive precursor to A, n is 0 or 1 and X is a leaving group, and wherein said agent of formula II or III having a percent incorporation of radioisotope * corresponding to the desired percent incorporation of the composition, or in excess thereof,
and in the case that agent of formula II or III having a percent incorporation of radioisotope * corresponding to the desired range, obtaining a product composition,
or alternatively in the case that agent of formula II or III having a percent incorporation of radioisotope * in excess of that desired,
obtaining a compound of formula I having percent incorporation of radioisotope * in excess, and additionally combining the obtained compound of formula I with an amount of B and obtaining product composition.

63. The process of claim 62, wherein a compound of formula II or III is a functionalized or unfunctionalized C₁₋₄ hydrocarbon, and wherein A′ or A is ¹⁴C or ³H C₁₋₄ hydrocarbyl or an unconjugated radioisotope, and X is selected from —C═O, —Br, —I, —Cl, —OH, or is an unconjugated atom.

64. The process of claim 62, wherein a compound of formula II or III is ¹⁴C or ³H alkene, formaldehyde, acetaldehyde or any other methylating agent as known in the art or is ³⁶Cl chlorine gas (Cl₂).

65. The process of claim 62, wherein said reactive agent is a maleimide.

66. The process of claim 62, wherein said reactive agent is ¹⁴C—N-ethyl maleimide.

67. The process of claim 62, further comprising the steps of: labelling an agent II or III or labelling a compound B to give a compound of formula I;
determining the specific activity thereof;
determining the desired specific activity to give a desired percent incorporation;
combining said agent or compound with a sufficient amount of corresponding unlabeled agent or compound of formula I or biological B; and
isolating the same as a homogeneous product or as a composition having the desired percent incorporation.

68. A method for AMS detection of one or more biological compositions B as defined in claim 39, wherein biological composition B is of the same or different origin, comprising the steps of:
1) providing one or more biological compositions B and optionally one or more control compositions comprising biological compound B;
2) dosing at least one subject with at least one biological composition B;
3) optionally dosing at least one subject with one or more control compositions comprising biological compound B,
4) obtaining metabolic samples from the at least one subject dosed with said biological composition B, and optionally the at least one subject dosed with said control compositions comprising biological compound B;
5) conducting AMS detection on said metabolic samples from step 4);
6) obtaining AMS results for the samples from step 5) and
7) correlating the AMS results from said samples from step 5) to the least one biological composition B, and optionally to at least one subject dosed with said control compositions comprising biological compound B.

69. The method according to claim 68, wherein said subject is any human, animal or plant or is an assay medium or cell culture.

70. The method according to claim 68, wherein said method for AMS detection of a biological composition B, comprising the additional steps of:
8) correlating the binding to target, metabolic fate, ADME, and PK data of the at least one biological composition B to the AMS results of said biological composition B;
9) optionally correlating the binding to target, metabolic fate, ADME, and PK data of the control composition comprising biological composition B to the AMS results of said control biological composition B; and
10) comparing the results of step 8) with those of step 9).

71. A method for determining the bioequivalence of two or more biological compositions B derived from same or different origin for product control and establishing reproducibility of the source of biological B, wherein said method comprises the steps of:
1) obtaining a first biological composition B;
2) performing the method of AMS detection according to claim 68 on said first biological composition B derived from a first source;
3) performing the method of AMS detection according to claim 68 on at least a second biological composition B derived from one or more different sources;
4) comparing the AMS results from said first biological composition B derived from a first source with the AMS results from the at least the second biological composition B from one or more different sources; and
5) determining whether the at least second biological composition B is bioequivalent to said first biological composition B.

72. The method according to claim 71, comprising the additional step of comparing AMS results for each biological B with its corresponding composition.

73. The method according to claim 71, wherein comparing AMS results determines whether said first and second biological compositions B are identical, and if there are any differences, whether said differences are the consequence of bio non-equivalence or of sample or host variation which are irrelevant to bio equivalence.

74. The method according to claim 71, wherein said method of performing AMS detection in step 2) comprises the additional steps of:
8) correlating the binding to target, metabolic fate, ADME, and PK data of the at least one biological composition B to the AMS results of said biological composition B;
9) optionally correlating the binding to target, metabolic fate, ADME, and PK data of the control composition comprising biological composition B to the AMS results of said control biological composition B; and
10) comparing the results of step 8) with those of step 9).

75. The method according to claim 73, wherein the source of said first biological composition B is an endogenous human source and the source of said at least second biological composition B is an animal biological B.

76. The method according to claim 73, wherein the source of said first biological composition B is an endogenous human source and the source of said at least second biological composition B is a genetically engineered biological B.

77. A method for ensuring that a generic or pirate biological composition B is not wrongly substituted for a proprietary biological composition B, according to claim 73, wherein the source of said first biological composition B is a proprietary biological B and the source of said at least second biological composition B is a generic or pirate biological B.

78. A method of use of a biological composition B, or compound of formula I, in accordance with the method of AMS detection of claim 68, in order to determine in vitro or in vivo metabolism characteristics of said biological composition B, or compound of formula I.


SEQUENCE LISTING (1 sequence)

SEQ ID NO: 1 - protein, 21 aa, Homo Sapiens
  Homo Sapiens: N-ethyl maleimide fragment of Human serum albumin from tryptic digest
ALVLIAFAQYLQQCPFEDHVK